(19) United States
(12) Patent Application Publication
(10) Pub. No.: US 2026/0264515 A1
(43) Pub. Date: Sep. 10, 2026

(54) ALCOHOL MEASUREMENT APPARATUS AND METHOD THEREOF

(71) Applicants: Hyundai Motor Company, Seoul (KR); Kia Corporation, Seoul (KR)

(72) Inventors: Eung Hwan KIM, Hwaseong-Si (KR); Seul Ki JEON, Hwaseong-Si (KR); Hui Won SHIN, Hwaseong-Si (KR)

(21) Appl. No.: 19/284,097

(22) Filed: Jul. 29, 2025

(30) Foreign Application Priority Data
Mar. 7, 2025 (KR) 10-2025-0030082

(51) Int. Cl. B60K 28/06; G01N 33/00; G01N 33/497

(52) U.S. Cl. CPC B60K 28/063; G01N 33/0047; G01N 33/006; G01N 33/0068; G01N 33/0073; G01N 33/4972

(57) ABSTRACT

An alcohol measurement apparatus and a method thereof are provided. The alcohol measurement apparatus includes an inlet formed in a vehicle and configured to receive a first gas in the vehicle; a shutter provided on the inlet and configured to open to allow intake of the first gas through the inlet or close to prevent intake of the first gas through the inlet; an alcohol sensor connected, via a pipe, to the inlet and configured to measure alcohol content of the first gas received via the inlet; and a control circuit provided in the vehicle and configured to control, based on a predetermined measurement condition, the shutter to open or close.

### CROSS-REFERENCE TO RELATED APPLICATION

This application claims the benefit of and priority to Korean Patent Application No. 10-2025-0030082, filed in the Korean Intellectual Property Office on March 7, 2025, the entire contents of which are incorporated herein by reference.

### TECHNICAL FIELD

The present disclosure relates to vehicle safety devices, and more specifically an alcohol measurement apparatus and a method thereof.

### BACKGROUND

An alcohol detection device (e.g., an alcohol sensor) in a vehicle may come in different varieties. For example, an external accessory (e.g., add-on) type may have a structure for communicating with the power source via the wire. Because the external accessory type of alcohol detection device may require a driver take out and hold the drinking measurement sensor and exhale into the drinking measurement sensor, it may be inconvenient to use and detract from the aesthetics in the vehicle.

If the sensor is placed in the vehicle in a vehicle driving environment, because of contamination (e.g., collection of dust particles) and condensation (e.g., collection of moisture) in the sensor that either directly absorbs respiratory gases or indirectly analyzes the respiratory gases, the longevity and the accuracy of measurement of the sensor may be degraded.

If the driver attempts to tamper with the measurement device, for example, by inserting foreign substances or liquids into the sensor, for example, to avoid detection, the alcohol content measurement may be further hindered.

Thus, there exists a need for a technology for more effective alcohol measurement in a vehicle.

### SUMMARY

The present disclosure has been made to solve the above-mentioned problems occurring in at least some implementations while advantages achieved by those implementations are maintained intact.

An aspect of the present disclosure provides a measurement apparatus using an inlet formed in a vehicle and a method thereof.

Another aspect of the present disclosure is to configure a sensor in the form of being embedded in a built-in structure in a vehicle and an inlet for introducing respiratory gases to facilitate drinking measurement in the vehicle.

The technical problems to be solved by the present disclosure are not limited to the aforementioned problems, and any other technical problems not mentioned herein will be clearly understood from the following description by those skilled in the art to which the present disclosure pertains.

According to one or more example embodiments of the present disclosure, an alcohol measurement apparatus may include: an inlet formed in a vehicle and configured to receive a first gas in the vehicle; a shutter provided on the inlet and configured to open to allow intake of the first gas through the inlet or close to prevent intake of the first gas through the inlet; an alcohol sensor connected, via a pipe, to the inlet and configured to measure alcohol content of the first gas received via the inlet; and a control circuit provided in the vehicle and configured to control, based on a predetermined measurement condition, the shutter to open or close.

The alcohol measurement apparatus may further include: a moisture sensor provided in the pipe and configured to measure moisture content inside the pipe; and a drain provided in the pipe and configured to: open to drain moisture in the pipe, or close to prevent the moisture in the pipe from draining. The control circuit may be further configured to: control, based on the measured moisture content in the pipe, the drain to open or close.

The control circuit may be further configured to: determine whether the measured alcohol content is above a threshold level; and limit, based on the measured alcohol content being above the threshold level, starting or driving of the vehicle.

The control circuit may be further configured to: based on determining that the alcohol content measured at a first time is below a threshold level and detecting, after the first time, a driving pattern of the vehicle that is predetermined to be a careless driving pattern, control the alcohol sensor to measure, at a second time after the first time, second alcohol content of a second gas received via the inlet; and limit, based on the second alcohol content being above the threshold level, starting or driving of the vehicle.

The predetermined measurement condition may include: outputting, via a user interface of the vehicle while an ignition of the vehicle is on or the vehicle is operated to move from a stopped position, an alcohol measurement request message; and controlling the shutter to open.

The predetermined measurement condition may include: controlling, while the vehicle is moving, the shutter to open; and controlling the alcohol sensor to measure alcohol content of a second gas received via the inlet.

The predetermined measurement condition may include: determining, based on an image of a driver in the vehicle, whether it is possible to measure the alcohol content.

The control circuit may be further configured to: determine, based on a direction of a face of the driver in the image and a position of the face in the image, whether it is possible to measure the alcohol content.

The control circuit may be further configured to: determine, based on a change of a face of the driver in the image, whether it is possible to measure the alcohol content.

The control circuit may be further configured to: output, based on detecting a predetermined tool or shape in the image, an alarm indicating that it is impossible to measure the alcohol content; and control the shutter to close.

According to one or more example embodiments of the present disclosure, a method, performed by an alcohol measurement apparatus of a vehicle, may include: determining, based on sensing data of at least one sensor of the vehicle, whether a predetermined measurement condition is met; and controlling, based on whether the predetermined measurement condition is met, a shutter to open or close. The shutter may be provided on an inlet connected to an alcohol sensor. The inlet may be formed in the vehicle. The method may further include: measuring, via the alcohol sensor and based on the shutter being closed, alcohol content of a first gas received via the inlet; and outputting, based on the measured alcohol content, a signal indicating the measured alcohol content.

The method may further include: measuring, via a moisture sensor, moisture content in a pipe connecting the inlet to the alcohol sensor; and controlling, based on the measured moisture content, a drain to open or close.

The method may further include: determining whether the measured alcohol content is above a threshold level; and limiting, based on the measured alcohol content being above the threshold level, starting or driving of the vehicle.

The method may further include: based on determining that the alcohol content measured at a first time is below a threshold level and detecting, after the first time, a driving pattern of the vehicle that is predetermined to be a careless driving pattern, controlling the alcohol sensor to measure, at a second time after the first time, second alcohol content of a second gas received via the inlet; and limiting, based on the second alcohol content being above the threshold level, starting or driving of the vehicle.

The predetermined measurement condition may include: outputting, via a user interface of the vehicle while an ignition of the vehicle is on or the vehicle is operated to move from a stopped position, an alcohol measurement request message; and controlling the shutter to open.

The predetermined measurement condition may include: controlling, while the vehicle is moving, the shutter to open; and measuring, via the alcohol sensor, alcohol content of a second gas received via the inlet.

The predetermined measurement condition may include: determining, based on an image of a driver in the vehicle, whether it is possible to measure the alcohol content.

Determining of whether it is possible to measure the alcohol content may include: determining, based on a direction of a face of the driver in the image and a position of the face in the image, whether it is possible to measure the alcohol content.

Determining of whether it is possible to measure the alcohol content may include: determining, based on a change of a face of the driver in the image, whether it is possible to measure the alcohol content.

The method may further include: outputting, based on detecting a predetermined tool or shape in the image, an alarm indicating that it is impossible to measure the alcohol content; and controlling the shutter to close.

### BRIEF DESCRIPTION OF THE DRAWINGS

The above and other objects, features and advantages of the present disclosure will be more apparent from the following detailed description taken in conjunction with the accompanying drawings:

FIG. 1 is a block diagram illustrating a configuration of an example alcohol measurement apparatus;

FIG. 2 is a drawing illustrating a configuration of an inlet of an example alcohol measurement apparatus;

FIG. 3 is a drawing illustrating a configuration of an inlet and a sensor module of an example alcohol measurement apparatus;

FIG. 4 is a drawing illustrating a configuration of an inlet, a shutter, and a sensor module of an example alcohol measurement apparatus;

FIG. 5 is a drawing illustrating some components of an example alcohol measurement apparatus;

FIG. 6 is a flowchart illustrating an example alcohol measurement method performed in an alcohol measurement apparatus;

FIG. 7 is a flowchart illustrating an example alcohol measurement method performed in an alcohol measurement apparatus;

FIG. 8 is a flowchart illustrating an example alcohol measurement method performed in an alcohol measurement apparatus; and

FIG. 9 illustrates an example computing system.

### DETAILED DESCRIPTION

Hereinafter, one or more example embodiments of the present disclosure will be described in detail with reference to the drawings. In adding the reference numerals to the components of each drawing, it should be noted that the identical component is designated by the identical numerals even when they are displayed on other drawings. Further, in describing the example embodiment(s) of the present disclosure, a detailed description of well-known features or functions will be ruled out in order not to unnecessarily obscure the gist of the present disclosure.

In describing the components of the example embodiment(s) of the present disclosure, terms such as first, second, “A”, “B”, (a), (b), and the like may be used. These terms are only used to distinguish one component from another component, but do not limit the corresponding components irrespective of the order or priority of the corresponding components. Furthermore, unless otherwise defined, all terms including technical and scientific terms used herein have the same meaning as being generally understood by those skilled in the art to which the present disclosure pertains. Such terms as those defined in a generally used dictionary are to be interpreted as having meanings equal to the contextual meanings in the relevant field of art, and are not to be interpreted as having ideal or excessively formal meanings unless clearly defined as having such in the present application.

For purposes of the present application and the claims, using the exemplary phrase “at least one of: A; B; or C” or “at least one of A, B, or C,” the phrase means “at least one A, or at least one B, or at least one C, or any combination of at least one A, at least one B, and at least one C. Further, exemplary phrases, such as "A, B, or C", "at least one of A, B, and C", "at least one of A, B, or C", etc. as used herein may mean each listed item or all possible combinations of the listed items. For example, "at least one of A or B" may refer to (1) at least one A; (2) at least one B; or (3) at least one A and at least one B.

Unless otherwise defined, the terms used herein, including technical or scientific terms, may have meanings generally understood by those skilled in the art to which the present disclosure belongs.

The expressions such as “comprise,” “may comprise,” “include,” “may include,” “have,” “may have,” etc. as used herein are intended to mean the presence of a characteristic (e.g., function, operation, component, etc.) and do not exclude the presence of other additional characteristics. That is, these expressions should be understood as open-ended terms that encompass the possibility that other examples are included.

A singular expression used herein may include the meaning of the plural unless otherwise stated in the context, which also applies to the singular expression described in the claims.

Expressions such as “first” or “second” as used herein are used to distinguish one object from another in referring to multiple similar objects, unless otherwise indicated in context, and do not limit the order or importance between them. For example, a plurality of chips according to the present disclosure may be distinguished from each other by referring them as “first chip,” “second chip,” respectively.

The term “unit” as used herein may refer to software, or hardware component such as Field-Programmable Gate Array (FPGA), Application Specific Integrated Circuit (ASIC), etc. However, “unit” is not limited to hardware and software. The “unit" may be configured to be stored in an addressable storage medium, or may be configured to execute one or more processors. The "unit" may include components such as software components, object-oriented software components, class components, and task components, as well as processors, functions, attributes, procedures, subroutines, segments of program code, drivers, firmware, microcode, circuits, data, databases, data structures, tables, arrays, and variables.

The expression “based” on as used herein is intended to describe one or more factors that influence an act or operation of determining or deciding described in a phrase or sentence including that expression, and this expression does not exclude any additional factors that influence the act or operation of determining or deciding.

When it is described that a component (e.g., a first component) is “connected” or “coupled” to another component (e.g., a second component) as used herein, it may mean that the component is not only directly connected or coupled to another component, but also connected or coupled through yet another component (e.g., a third component).

Depending on the context, the expression “configured to” as used herein may have meanings such as “set to,” “with the ability to,” “modified to,” “made to,” “to be able to,” etc. This expression is not limited to the meaning of “specially designed in hardware to.” For example, a processor configured to perform a specific operation may refer to a generic purpose processor capable of performing the specific operation by executing software, or to a special purpose computer structured through programming to perform the specific operation.

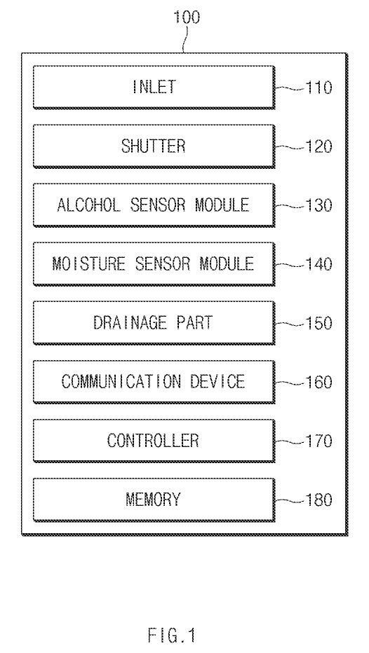

Hereinafter, one or more example embodiments of the present disclosure will be described in detail with reference to FIGS. 1 through 9.

FIG. 1 is a block diagram illustrating a configuration of an example alcohol measurement apparatus. Referring to FIG. 1, an alcohol measurement apparatus 100 may be a computable computing device. The alcohol measurement apparatus 100 may be loaded into a smartphone, a robot, a vehicle, a mobility device, and/or the like. For example, the alcohol measurement apparatus 100 may be implemented in a vehicle. In this case, the alcohol measurement apparatus 100 may be integrally configured with control units in the vehicle or may be implemented as a separate device to be connected with the control units of the vehicle by a separate connection means.

The alcohol measurement apparatus 100 may refer to a device for measuring alcohol in the vehicle. For example, the alcohol measurement apparatus 100 may be a device for measuring alcohol to determine whether a measurement target (e.g., a driver, a passenger, or the like) in the vehicle is in a drunken state (also referred to as a high alcohol state).

The alcohol measurement apparatus 100 may include an inlet (also referred to as an intake) 110, a shutter 120, an alcohol sensor module (also referred to as an alcohol sensor, an alcohol meter, or a breathalyzer) 130, a moisture sensor module (also referred to as a moisture sensor) 140, a drainage part (also referred to as drainage or a drain) 150, a communication device 160, a controller (also referred to as a control circuit) 170, a memory 180, and the like. The respective components of the alcohol measurement apparatus 100 may be connected with each other via in-vehicle communication. An inter-vehicle communication network may be implemented as at least one of communication technologies, such as a controller area network (CAN), a media oriented systems transport (MOST) network, a local interconnect network (LIN), and an X-by-Wire. The alcohol measurement apparatus 100 and the other components in the vehicle may be connected with each other via the in-vehicle communication. The other components in the vehicle may include an audiovisual guidance device (e.g., a user interface, a display device, a voice guidance device, or the like), a vehicle start and power system, a telematics system, an imaging device, and the like.

The imaging device may be mounted into the vehicle to determine whether a measurement behavior of a measurement target is appropriate and whether there is no dishonest behavior or an inappropriate factor in the measurement behavior of the measurement target. If normal drinking measurement (or normal alcohol measurement) is not performed, the imaging device may request remeasurement or may process it as a situation in which it is impossible to determine driving measurement. The imaging device may include an imaging device or a separate camera device to perform driver state monitoring in the vehicle. The imaging device may analyze image information input before and after driving measurement, thus improving the accuracy and reliability of the drinking measurement.

The imaging device may perform a driver check, a surrogate measurement check, drinking possibility determination, an honest measurement check, dishonest measurement determination, or the like.

The driver check may refer to checking driver’s sitting or a driver’s identity (ID). The driver check may refer to detecting a driver change (e.g., changing between the driver and the passenger, switching between the driver and the passenger, or the like), after measuring a non-drinker.

The surrogate measurement check may refer to checking whether another person except for the driver exists in a predetermined field of view (FoV).

The drinking possibility determination may refer to determining a possibility of drinking depending on whether a flush (or a red color) is discovered in the eyeball and face. The drinking possibility determination may refer to performing comparison with a past face upon driving to determine the possibility of drinking. The drinking possibility determination may refer to determining the possibility of drinking depending on whether the driver keeps his or her eyes on the road and performs a careless behavior.

The honest measurement check may refer to checking the direction of the face orients the inlet 110. For example, the honest measurement check may be checking whether the driver is facing the front with regard to whether both eyes are shown, whether left and right cheeks are shown, whether there is facial symmetry, or whether pupils are looking straight ahead.

The honest measurement check may refer to checking whether cheeks puff out and the mouth is opened at the moment of exhaling. For example, the honest measurement check may be checking a change in face inhaling to exhale (e.g., mouth opening and upper body movement) or a change (e.g., mouth protrusion and opening) in face when exhaling.

The honest measurement check may refer to checking whether the face position of the driver is a correct position. For example, the honest measurement check may be estimating a distance from a steering wheel base on a size of a head part and a face component (e.g., eyes, a nose, a mouth, ears, or the like) in an FoV of the imaging device to check whether the face position of the driver is the correct position.

The dishonest measurement determination may refer to checking whether a dishonest tool (e.g., a spray, a balloon, a spray hose, a fan type, or the like) is used.

The dishonest measurement determination may refer to checking whether the driver sprays liquid (e.g., whether the driver takes a spitting behavior, takes a behavior using a container (e.g., a bottle, a cup, or the like), or the like).

The dishonest measurement determination may refer to checking whether the driver ingests food and/or liquid.

The imaging device may be controlled by the controller 170. Thus, the controller 170 may perform the above-mentioned tasks via the imaging device.

The inlet 110 may be formed in the vehicle and may absorb gas (e.g., a first gas, a second gas, or the like) in the vehicle. The first gas may be gas absorbed by opening the shutter 120 after preparation for measurement is completed. The second gas may be gas absorbed by opening the shutter 120, while the vehicle is driving.

The inlet 110 may be located at a position where it is able to capture the respiration of a target to be measured as best as possible. The inlet 110 may be formed in a separate pipe or an instrument for guiding respiratory gases. For example, the inlet 110 may be formed in a side door, a front ceiling, a steering wheel, periphery of a dashboard, a cockpit (or the dashboard), an A or B filter, or the like of the vehicle.

The inlet 110 may be in the form of a “hole” or a “hole and snorkel” in the vehicle interior surface. The hole may include a grid or a vertical blade or bulkhead to minimize the amount of moisture being introduced and lateral respiration influences.

The shutter 120 may be provided on the inlet 110 and may be controlled to be opened or closed to absorb gas. The shutter may, for example, open to allow passage (e.g., intake) of the first gas through the inlet, or the shutter may close to prevent passage (e.g., intake) of the first gas through the inlet. The shutter 120 may be, for example, an adjustable louver that may be adjusted between an open position and a closed position to regulate air flow. For example, the controller 170 may open the shutter 120 during the moment when alcohol is measured by means of predetermined software logic, thus passing respiration of the measurement target (e.g., the driver). If the alcohol sensor module 130 and/or the moisture sensor module 140 becomes dusty or wet, because they may deteriorate in performance and durability, the shutter 120 may be always closed and the shutter 120 may be opened only at the moment of the measurement.

The shutter 120 may include one or more motors and an actuator, such as a piezo actuator or a solenoid actuator, and may open or close a ventilation hole using the actuator.

The alcohol sensor module 130 may be provided in the pipe connected with the inlet 110 and may measure alcohol content (e.g., breath alcohol content (BrAC)) from the first gas absorbed via the inlet 110.

The alcohol sensor module 130 may be a sensor capable of measuring alcohol from air in the vehicle or respiration of the measurement target (e.g., the driver, a passenger, or the like). A scheme of the alcohol sensor module 130 may include an optical scheme, an electrochemical scheme, a semiconductor scheme, or the like.

For example, the alcohol sensor module 130 may (e.g., directly) adsorb alcohol molecules (e.g., C2H5OH, CH4, or the like) in the air to measure a voltage and/or current change by a chemical reaction. For another example, the alcohol sensor module 130 may indirectly emit light to a determined space at an emitter and may measure reflection by a wavelength band of alcohol molecules at a receiver to calculate and measure the amount of alcohol occupied in the air.

The moisture sensor module 140 may be provided in the pipe connected with the inlet 110 and may measure moisture absorbed via the inlet 110 (e.g., moisture content in the pipe). The moisture sensor module 140 may detect moisture introduction via a sensor (e.g., a humidity sensor, an optical device, an electrode body, or the like) for sensing the moisture introduction. The controller 170 may close the shutter 120 upon the moisture introduction.

The amount of introduction for determining moisture introduction in the moisture sensor module 140 may vary with a shape and size of the inlet 110 in the steering wheel or an air flow duct (or a pipe).

The drainage part 150 may be provided in the pipe connected with the inlet 110 and may be controlled to be opened or closed to drain moisture which is present on the pipe connected with the inlet 110.

If moisture is introduced via an air intake or outlet, the drainage part 150 may play a role in draining the moisture. If moisture is introduced into an airbag module, a horn switch, a manipulation switch, various wires, or the like, because it is able to have a harmful influence on driving safety, there is a need to block moisture introduction. Moisture may be introduced in a vehicle usage environment, such as fluid intake of a driver, drink spilling of the driver, or usage of a sprayer of the driver. A drainage pipe (or a passage toward a liquid outlet) may be configured as a path in which moisture does not come into contact with the sensor.

The drainage part 150 may be located to drain moisture to the outside of the sensor or the air flow duct (or the pipe) and may process moisture in a method, such as indoor drop discharge, discharge outside the vehicle, natural evaporation, or separate container storage.

The communication device 160 may include any wired and wireless communication network capable of transmitting and receiving any type of data, information, signal, and the like. The communication device 160 may assist in transmitting and receiving data, information, a signal, and the like with an external device. The external device may be, for example, a device inside the vehicle, a device outside the vehicle, or the like.

The processor 170 may be composed of one or more cores and may include a controller for performing an operation associated with data processing, for example, a central processing unit (CPU), a general purpose graphics processing unit (GPGPU), or a tensor processing unit (TPU) of the alcohol measurement apparatus 100.

The controller 170 may usually control the overall operation of the alcohol measurement apparatus 100. The processor 170 may process a signal, data, information, or the like input or output via the components included in the alcohol measurement apparatus 100 or may run an application program stored in the memory 180, thus providing or processing appropriate information or an appropriate function to a user.

The controller 170 may be provided in the vehicle and may control the shutter 120 to be opened or closed depending on whether a predetermined measurement condition is met.

The controller 170 may control the drainage part 150 to be opened or closed, based on the result of measuring the moisture. For example, if the amount of moisture according to the result of measuring the moisture is greater than or equal to a predetermined amount (e.g., 2 cc or the like), the controller 170 may control the drainage part 150 to be opened, such that the drainage part 150 processes moisture in the method, such as indoor drop discharge, discharge outside the vehicle, natural evaporation, or separate container storage.

The controller 170 may determine whether a measurement target (e.g., a driver of the vehicle) in the vehicle is in a drunken state (e.g., above a threshold alcohol content level) based on the result of measuring the alcohol. For example, if a blood alcohol concentration according to the result of measuring the alcohol is greater than or equal to a predetermined concentration (e.g., 0.03%), the controller 170 may determine that the measurement target is in the drunken state.

If it is determined that the measurement target is in the drunken state, the controller 170 may limit (e.g., regulate, prevent, etc.) an operation (e.g., starting or driving) of the vehicle.

If it is determined that the measurement target is in the drunken state, the controller 170 may continuously provide the measurement target with a warning alarm.

If it is determined that the measurement target is in the drunken state, the controller 170 may transmit measured information and/or an indoor image to a server or a user terminal of a specific person (e.g., a protector, a supervisor, a specified person, or the like) via telematics.

If it is determined that the measurement target is not in the drunken state, the controller 170 may remeasure alcohol after a predetermined time (e.g., 10 minutes). For example, if it is determined that the measurement target is not in the drunken state, the controller 170 may control the shutter 120 to be opened after the predetermined time and may remeasure alcohol from air newly absorbed via the inlet 110.

The controller 170 may remeasure alcohol, until the operation of the vehicle ends (e.g., until the vehicle is ignition off).

If the vehicle is ignition on or is trying to move in a state in which the vehicle is stopped, the controller 170 may provide the measurement target in the vehicle with guidance (e.g., a message, an announcement, instructions, etc.) for requesting alcohol measurement and may control the shutter 120 to be opened. For example, an alcohol measurement request message may be output (e.g., if an ignition of the vehicle is on and/or the vehicle is operated to move from a stopped position) via a user interface of the vehicle.

If the vehicle is driving, the controller 170 may control the shutter 120 to be opened and may measure alcohol from a second air absorbed via the inlet 110, using the alcohol sensor module 130.

The controller 170 may determine whether it is possible to measure alcohol based on an image of the measurement target, which is captured in the vehicle.

For example, the controller 170 may determine whether it is possible to measure alcohol based on a face direction and a position of the measurement target in the image.

For another example, the controller 170 may determine whether it is possible to measure alcohol based on a change in face of the measurement target in the image.

For another example, if a predetermined tool or shape is recognized in the image, the controller 170 may provide the measurement target with an alarm indicating that it is impossible to measure the alcohol and may control the shutter 120 to be closed.

The memory 180 may store any type of information generated or determined by the controller 170 and/or any type of information received by the communication device 160. For example, the memory 180 may store pieces of information associated with alcohol measurement, pieces of information associated with moisture measurement, or the like. The memory 180 may operate by the processor 170. The memory 180 may store software programmed such that the controller 170 performs a determined operation.

The memory 180 may include at least one type of storage medium among a flash memory type memory, a hard disk type memory, a multimedia card micro type memory, a card type memory (e.g., an SD or XD memory), a random access memory (RAM), a static RAM (SRAM), a read-only memory (ROM), an electrically erasable programmable ROM (EEPROM), a programmable ROM (PROM), a magnetic memory, a magnetic disc, and/or an optical disc. The alcohol measurement apparatus 100 may operate in conjunction with web storage for performing a storage function of the memory 180 on the Internet. The above-mentioned description of the memory 180 is only an example and the present disclosure is not limited thereto.

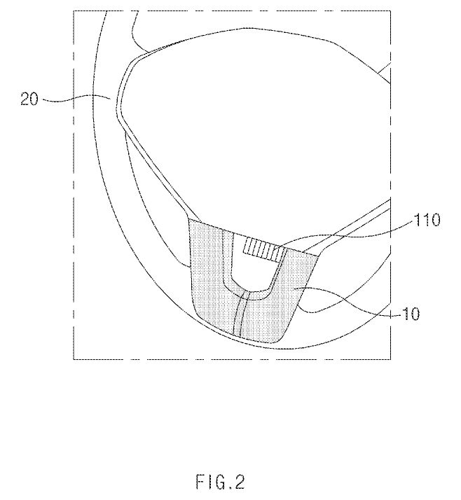

FIG. 2 is a drawing illustrating a configuration of an inlet of an example alcohol measurement apparatus.

Referring to FIG. 2, an inlet 110 may be formed in a steering wheel 20 of a vehicle. Because the distance from a nose and/or a mouth which are/is a respiratory portion of a measurement target to the steering wheel 20 is generally within 1 M, the steering wheel 20 among structures in the vehicle may have the shortest distance from the respiratory portion.

An alcohol measurement apparatus 100 may further include a funnel structure 10 or a structure with an inclined surface to guide flow of the air to be measured to the inlet 110.

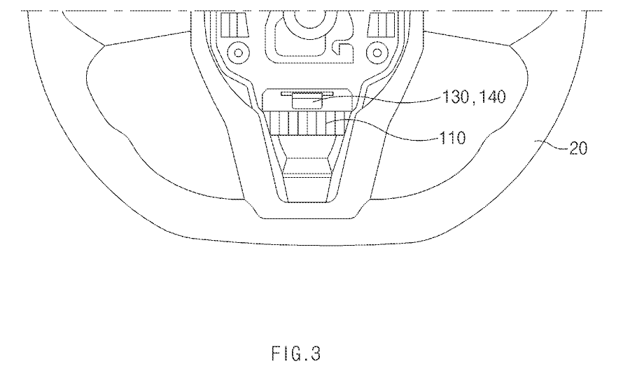

FIG. 3 is a drawing illustrating a configuration of an inlet and a sensor module of an example alcohol measurement apparatus.

Referring to FIG. 3, an inlet 110 is formed in a steering wheel 20 of a vehicle. An alcohol sensor module 130 and/or a moisture sensor module 140 may be provided in the steering wheel 20.

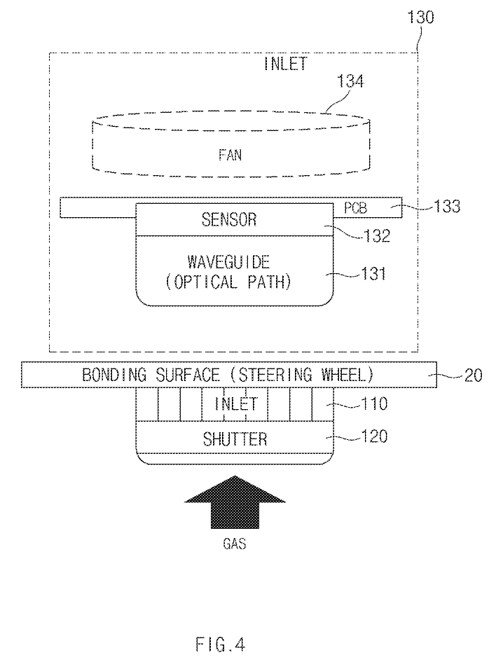

FIG. 4 is a drawing illustrating a configuration of an inlet, a shutter, and a sensor module of an example alcohol measurement apparatus.

Referring to FIG. 4, an inlet 110 may be formed in a steering wheel 20 of a vehicle. The inlet 110 may be composed of a ventilation hole, which is open toward the outside of the steering wheel 20, and a shutter 120 to introduce air. The shutter 120 may be provided on the inlet 110 and may be controlled to be opened or closed to absorb air. The shutter 120 may be included in the inlet 110 or may be provided to be separately attachable or detachable.

An alcohol sensor module 130 may include a waveguide (or an optical path) 131 for delivering light, an optical sensor 132, a printed circuit board (PCB) 133, a fan 134 or suction machine for inhaling or exhaling gas, and the like.

If there is a lack of the amount of introduced air, the fan 134 or the suction machine may operate to increase the amount of gas inhalation.

The alcohol sensor module 130 may indirectly emit light to a determined space at an emitter using the above-mentioned components and may measure reflection by a wavelength band of alcohol molecules at a receiver, thus calculating the amount of alcohol occupied in the air and measuring alcohol.

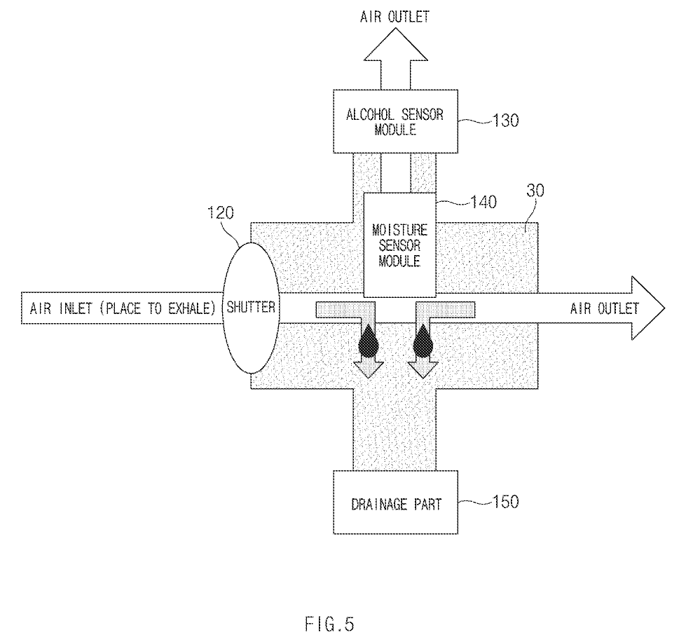

FIG. 5 is a drawing illustrating some components of an example alcohol measurement apparatus.

Referring to FIG. 5, a pipe 30 may be in the shape of a hollow tube, which may include an intake in which gas and/or liquid are/is inhaled, a gas outlet for exhaling the inhaled gas, and a liquid outlet for exhaling the inhaled liquid.

An inlet 110 and a shutter 120 may be provided in the intake and/or a passage toward the intake.

The gas outlet may pass sensor detection or may be composed of a separate ventilation hole and may play a role in exhaling exhale input for measurement. For example, an alcohol sensor module 130 and/or a moisture sensor module 140 may be provided in a passage toward a first gas outlet. For another example, a passage toward a second gas outlet may be formed independently of the passage toward the first gas outlet.

A drainage part 150 may be provided in the liquid outlet and/or the passage toward the liquid outlet.

If respiration of the measurement target is measured several times, because a measured value is inaccurate if the remaining respiration which is previous measured remains in the pipe 30, the alcohol sensor module 130, and/or the moisture sensor module 140, there may be a need for an outlet (e.g., the gas outlet, the liquid outlet, or the like) capable of exhaling the remaining respiration.

The shutter 120 may cause flow of gas to occur. For example, the shutter 120 may simultaneously open the intake and the gas outlet.

If the shutter 120 is opened, the drainage part 150 may be closed to prevent gas from being exhaled. If moisture is introduced into the pipe 30, the drainage part 150 may be opened while the shutter 120 is closed.

If using the moisture sensor module 140 and if detecting moisture introduction of the pipe 30 via the moisture sensor module 140, the controller 170 may automatically close the shutter 120.

The controller 170 may set a time when the shutter 120 is opened and closed during a continuous exhale time of the measurement target, thus minimizing influence of the air in the mouth and non-respiratory gases of the measurement target.

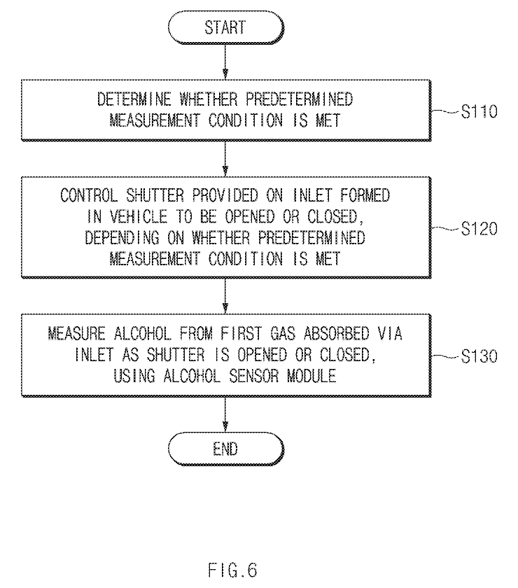

Hereinafter, an alcohol measurement method of the present disclosure will be described in detail with reference to FIGS. 6 to 8. FIGS. 6 to 8 are flowcharts illustrating an alcohol measurement method performed in an alcohol measurement apparatus.

Hereinafter, it is assumed that an alcohol measurement apparatus 100 of FIG. 1 performs example processes of FIGS. 6 through 8. Furthermore, in descriptions of FIGS. 6 through 8, it may be understood that an operation described as being performed by an apparatus is controlled by a processor 170 of the alcohol measurement apparatus 100. In the descriptions of FIGS. 6 through 8, a detailed description of the components of the alcohol measurement apparatus 100 may be replaced with the contents described above with reference to FIGS. 1 through 5.

FIG. 6 is a flowchart illustrating an example alcohol measurement method performed in an alcohol measurement apparatus.

Referring to FIG. 6, in S110, an alcohol measurement apparatus 100 may determine whether a predetermined measurement condition is met.

The alcohol measurement apparatus 100 may determine whether it is possible to measure alcohol based on an image of a measurement target, which is captured in a vehicle.

For example, the alcohol measurement apparatus 100 may determine whether it is possible to measure the alcohol based on a face direction and a position of the measurement target in the image.

For another example, the alcohol measurement apparatus 100 may determine whether it is possible to measure the alcohol based on a change in face of the measurement target in the image.

For another example, if a predetermined tool or shape is recognized in the image, the alcohol measurement apparatus 100 may provide the measurement target with an alarm indicating that it is impossible to measure the alcohol. For example, the alcohol measurement apparatus 100 may control a shutter 120 to be closed. The predetermined tool may be a dishonest tool. The predetermined shape may be a shape of the dishonest tool.

In S120, the alcohol measurement apparatus 100 may control the shutter 120 provided on an inlet 110 formed in the vehicle to be opened or closed, depending on whether the predetermined measurement condition is met.

For example, if the vehicle is ignition on or is trying to move in a state in which the vehicle is stopped, the alcohol measurement apparatus 100 may provide the measurement target in the vehicle with guidance (e.g., a message, an announcement, instructions, etc.) for requesting alcohol measurement. The alcohol measurement apparatus 100 may control the shutter 120 to be opened.

For another example, if the vehicle is driving, the alcohol measurement apparatus 100 may control the shutter 120 to be opened. The alcohol measurement apparatus 100 may measure alcohol from a second gas absorbed via the inlet 110 using an alcohol sensor module 130.

In S130, the alcohol measurement apparatus 100 may measure alcohol from a first gas absorbed via the inlet 110 as the shutter 120 is opened or closed, using the alcohol sensor module 130.

The alcohol measurement apparatus 100 may measure moisture absorbed via the inlet 110, using a moisture sensor module 140.

The alcohol measurement apparatus 100 may control a drainage part 150 for draining moisture to be opened or closed, based on the result of measuring the moisture.

The alcohol measurement apparatus 100 may determine whether the measurement target in the vehicle is in a drunken state based on the result of measuring the alcohol.

If it is determined that the measurement target is in the drunken state, the alcohol measurement apparatus 100 may limit starting or driving of the vehicle.

If the measurement target corresponds to at least one of a specific behavior or a careless driving state of the measurement target, after a predetermined time, if it is determined that the measurement target is not in the drunken state, the alcohol measurement apparatus 100 may remeasure alcohol.

The specific behavior of the measurement target may include a behavior of lowering the head, a behavior of continuously closing the eyes, or the like.

The careless driving state may include a state in which the measurement target does not keep his or her eyes on the road, a state in which the body of the measurement target continuously is out of a certain range, or the like.

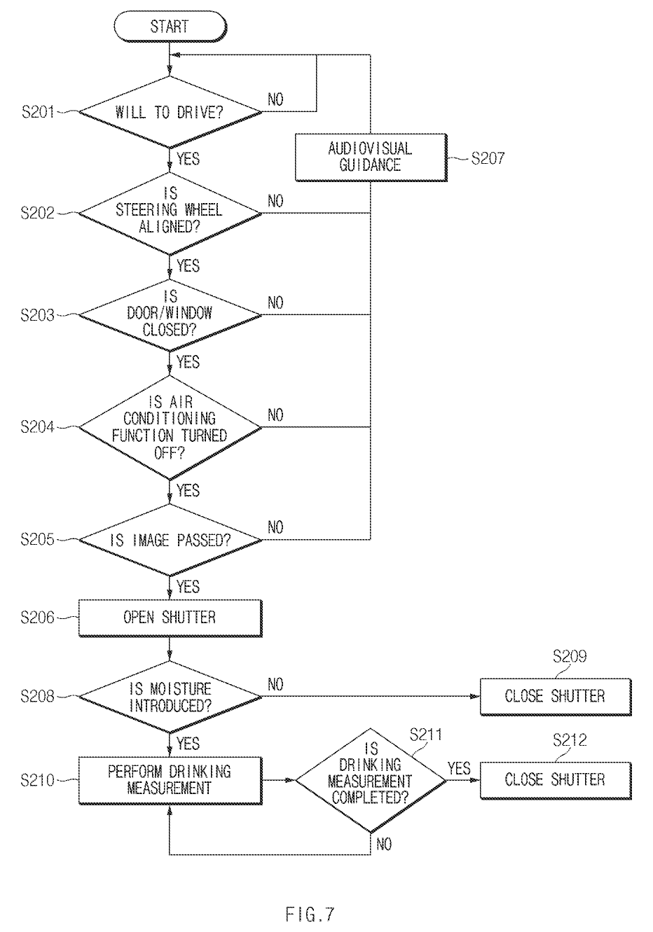

FIG. 7 is a flowchart illustrating an example alcohol measurement method performed in an alcohol measurement apparatus.

Referring to FIG. 7, in S201, an alcohol measurement apparatus 100 may determine whether a measurement target has a will to drive a vehicle (e.g., whether the driver attempts or is attempting to operate the vehicle). For example, if the measurement target changes a vehicle to a driving state, for example, if the measurement target pushes the start button of the vehicle, steps on the brake, or shifts to drive, the alcohol measurement apparatus 100 may determine that the measurement target has the will to drive.

If it is determined that the measurement target has the will to drive, the alcohol measurement apparatus 100 may determine whether the steering wheel of the vehicle is aligned. For example, if the steering wheel is not turned in a left or right direction, the alcohol measurement apparatus 100 may determine that the steering wheel is aligned. If the steering wheel is not aligned, the alcohol measurement apparatus 100 may control the steering wheel to be automatically aligned.

If it is determined that the steering wheel is aligned, in S203, the alcohol measurement apparatus 100 may determine whether a door and a window of the vehicle are closed. If the door and the window are not closed, the alcohol measurement apparatus 100 may control the open door and/or window to be closed.

If it is determined that the door and the window are closed, in S204, the alcohol measurement apparatus 100 may determine whether an air conditioning function of the vehicle is turned off. If the air conditioning function is not turned off, the alcohol measurement apparatus 100 may control to block the air conditioning function.

If it is determined that the air conditioning function is turned off, in S205, the alcohol measurement apparatus 100 may determine whether the image of the measurement target, which is captured in the vehicle, is passed. For example, if a face direction of the measurement target, a position of the measurement target, a change in the measurement target, or the like in the image is not matched with a predetermined passing condition, the alcohol measurement apparatus 100 may determine that the image is not passed.

If the component of the vehicle is not matched with the measurement condition in any one of S202, S203, S204, and S205 described above (NO), in S207, the alcohol measurement apparatus 100 may provide the measurement target with audiovisual guidance (e.g., message, announcement, instructions, etc.) via an audiovisual guidance device.

The audiovisual guidance device may include a cluster, audio video navigation (AVN), a head-up display (HUD), a display, a speaker, a buzzer, or the like, which is loaded into the vehicle.

If the steering wheel is not aligned, the audiovisual guidance device may provide the measurement target with guidance information to align the steering wheel to a correct position for driving straight.

If the door or the window is open, the audiovisual guidance device may provide the measurement target with guidance information to close the door, the window, or a sunroof.

If the air conditioning function is turned on, the audiovisual guidance device may provide the measurement target with guidance information to turn off the air conditioning function.

The face direction of the measurement target, the position of the measurement target, the change in the measurement target, or the like is not matched with the predetermined passing condition, the audiovisual guidance device may provide the measurement target with guidance information to keep his or her eyes on the road, guidance information to inhale the wind and then strongly exhale, guidance information to closely approach the steering wheel, or the like.

The pieces of guidance information described above may be obtained via a communication signal of each control unit, such as a body control unit (BCM) or driver state monitoring (DSM) in the vehicle.

If the component of the vehicle is not matched with the measurement condition in any one of S202, S203, S204, and S205 (NO), the vehicle may control its component to be matched with the measurement condition.

If the steering wheel is not aligned, the vehicle may align the steering wheel to a correct position for driving straight.

If the door or the window is open, the vehicle may close the door, the window, or the sunroof.

If the air conditioning function is turned on, the vehicle may pause the air conditioning function.

As described herein, the vehicle may be controlled by the alcohol measurement apparatus 100. Furthermore, the vehicle may automatically perform the operations as described herein.

If the components of the vehicle are matched with the measurement condition in S202, S203, S204, and S205 described above (YES), in S206, the alcohol measurement apparatus 100 may open a shutter 120.

In S208, the alcohol measurement apparatus 100 may determine whether moisture is introduced using a moisture sensor module 140.

If the moisture is introduced (S208 - NO), in S209, the alcohol measurement apparatus 100 may close the shutter 120. If the moisture is introduced, the alcohol measurement apparatus 100 may close the shutter 120 to block additionally moisture introduction.

If the moisture is not introduced (S208 - YES), in S209, the alcohol measurement apparatus 100 may perform drinking measurement. For example, the alcohol measurement apparatus 100 may measure alcohol in gas, using an alcohol sensor module 130.

In S211, the alcohol measurement apparatus 100 may determine whether the drinking measurement is completed. For example, if obtaining the amount of alcohol in the gas via the alcohol sensor module 130, the alcohol measurement apparatus 100 may determine that the drinking measurement is completed. For example, if the amount of alcohol in the gas is not obtained, the alcohol measurement apparatus 100 may determine that the drinking measurement is not completed.

If the drinking measurement is not completed (S211 - NO), in S210, the alcohol measurement apparatus 100 may perform drinking measurement again.

If the drinking measurement is completed (S211 - YES), in S212, the alcohol measurement apparatus 100 may close the shutter 120. The alcohol measurement apparatus 100 may complete the drinking measurement and may close the shutter 120, thus preventing pollution of the sensor due to dust and moisture, which are introduced via the shutter 120.

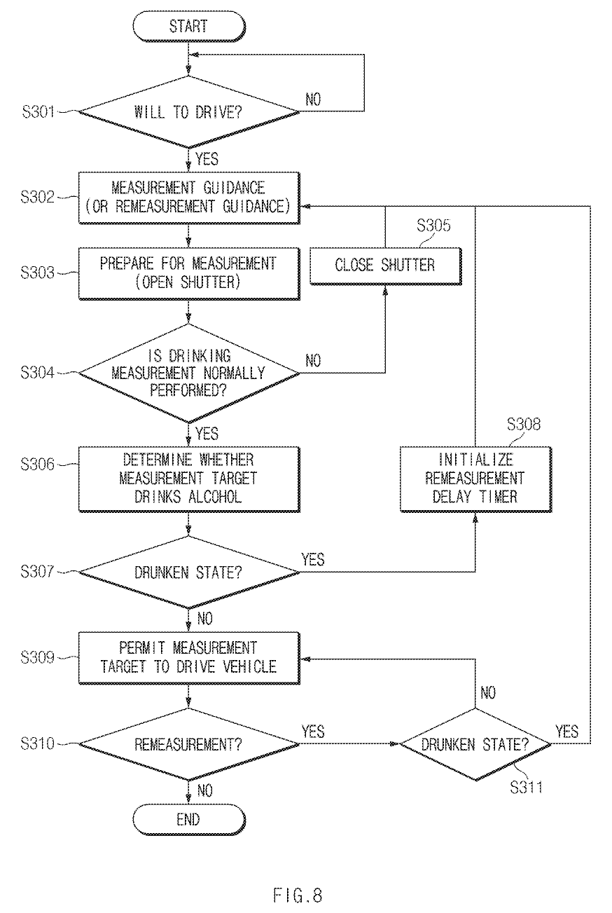

FIG. 8 is a flowchart illustrating an example alcohol measurement method performed in an alcohol measurement apparatus.

Referring to FIG. 8, in S301, an alcohol measurement apparatus 100 may determine whether a measurement target has a will to drive a vehicle. For example, if the measurement target changes a vehicle to a driving state, for example, if the measurement target pushes the start button of the vehicle, steps on the brake, or shifts to drive, the alcohol measurement apparatus 100 may determine that the measurement target has the will to drive.

If it is determined that the measurement target has the will to drive, in S302, the alcohol measurement apparatus 100 may provide the measurement target with measurement guidance (or remeasurement guidance). The alcohol measurement apparatus 100 may visually and audibly notify the measurement target that drinking measurement is initiated. For example, the alcohol measurement apparatus 100 may provide the measurement target with indication and auditory information for notifying the measurement target of exhaling or that there is a measurement mode.

In S303, the alcohol measurement apparatus 100 may prepare for measurement (e.g., open a shutter 120 or the like). The alcohol measurement apparatus 100 may prepare a vehicle and a sensor to accurately determine whether the measurement target (e.g., the driver) has consumed alcohol. For example, the alcohol measurement apparatus 100 may align a steering wheel of the vehicle or may guide the measurement target to align the steering wheel of the vehicle. The alcohol measurement apparatus 100 may control to close a door and a window of the vehicle. The alcohol measurement apparatus 100 may prohibit the measurement target from using certain objects prohibited from use before measurement, such as washer fluid (e.g., a mouth wash) or an air freshener and may operate an imaging device in the vehicle. The alcohol measurement apparatus 100 may warm up a sensor. The alcohol measurement apparatus 100 may perform zero-point calibration of the sensor. The alcohol measurement apparatus 100 may open an inlet 110 and the shutter 120.

In S304, the alcohol measurement apparatus 100 may check whether the drinking measurement is normally performed. The alcohol measurement apparatus 100 may determine the amount of respiration, carbon dioxide, the amount of alcohol, or the like. The alcohol measurement apparatus 100 may identify whether the drinking measurement is correct via the imaging device.

If introduction of moisture, smoke, dust, or the like is detected, in S305, the alcohol measurement apparatus 100 may close the shutter 120. The alcohol measurement apparatus 100 may stop the measurement and may provide the measurement target with remeasurement guidance in S302.

If the drinking measurement is normally performed, in S306, the alcohol measurement apparatus 100 may determine whether the measurement target drinks alcohol. The alcohol measurement apparatus 100 may determine whether the measurement target drinks alcohol using an alcohol sensor module 130.

If it is determined that the measurement target is in a drunken state (S307 - YES), in S308, the alcohol measurement apparatus 100 may initialize a remeasurement delay timer. The alcohol measurement apparatus 100 may provide the measurement target with the remeasurement guidance in S302.

If it is determined that the measurement target is not in the drunken state (S307 - NO), in S309, the alcohol measurement apparatus 100 may permit the measurement target to drive the vehicle.

In S310, the alcohol measurement apparatus 100 may determine whether to perform remeasurement. For example, the alcohol measurement apparatus 100 may perform any measurement, while the vehicle is driving, or may perform remeasurement, when a validity time expires (S310 - YES). For another example, if the driving of the vehicle ends, the alcohol measurement apparatus 100 may fail to perform remeasurement (S310 - NO).

The remeasurement delay timer may refer to a timer function for performing measurement or an operation again, after a certain time elapses from the measurement. Thus, if the measurement target is not in the drunken state, the alcohol measurement apparatus 100 may perform remeasurement after a certain time (e.g., the validity time) elapses using the remeasurement delay timer.

The alcohol measurement apparatus 100 may allow the measurement target to stop the vehicle and perform measurement or may measure indoor air or the measurement target using a sensor module, while the vehicle is driving.

The alcohol measurement apparatus 100 may determine a remeasurement period depending to an operation situation (e.g., a driving time or insufficiency, detection, and/or the like of a specific behavior (e.g., a driving posture, looking ahead, lane following, or the like).

After the remeasurement is performed (S310 - YES), in S311, the alcohol measurement apparatus 100 may determine whether the measurement target is in the drunken state. If it is determined that the measurement target is in the drunken state (S311 - YES), the alcohol measurement apparatus 100 may provide the measurement target with remeasurement guidance (e.g., an audiovisual information output for remeasurement request or the like) in S302.

If it is determined that the measurement target is not in the drunken state (S311 - NO), in S309, the alcohol measurement apparatus 100 may permit the measurement target to drive the vehicle.

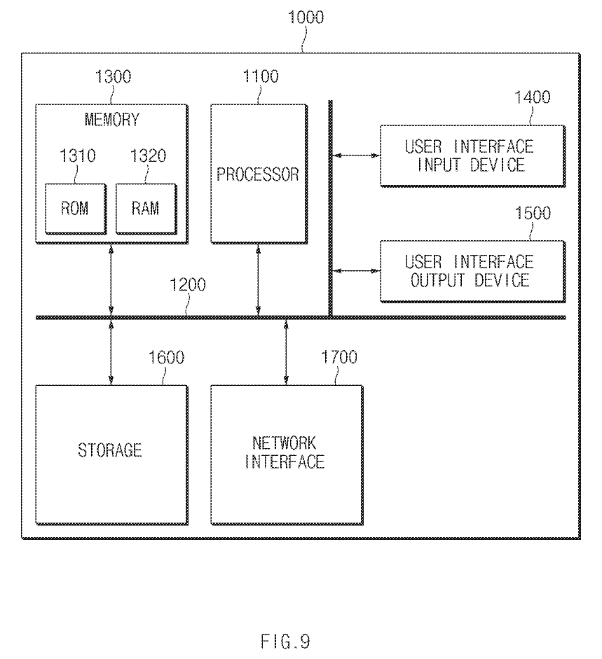

FIG. 9 illustrates an example computing system. The computing system may correspond to one or more of the components of an alcohol measurement apparatus.

Referring to FIG. 9, a computing system 1000 may include at least one processor 1100, a memory 1300, a user interface input device 1400, a user interface output device 1500, a storage 1600, and a network interface 1700, which are connected with each other via a bus 1200.

The processor 1100 may be a central processing unit (CPU) or a semiconductor device that processes instructions stored in the memory 1300 and/or the storage 1600. The memory 1300 and the storage 1600 may include various types of volatile or non-volatile storage media. For example, the memory 1300 may include a read only memory (ROM) 1310 and a random access memory (RAM) 1320.

Accordingly, the operations of the method or algorithm described in connection with the one or more embodiments disclosed in the specification may be directly implemented with a hardware module, a software module, or a combination of the hardware module and the software module, which is executed by the processor 1100. The software module may reside on a storage medium (i.e., the memory 1300 and/or the storage module 1600) such as a RAM, a flash memory, a ROM, an EPROM, an EEPROM, a register, a hard disc, a removable disk, and a CD-ROM.

The exemplary storage medium may be coupled to the controller, and the controller may read information out of the storage medium and may record information in the storage medium. Alternatively, the storage medium may be integrated with the controller. The controller and the storage medium may reside in an application specific integrated circuit (ASIC). The ASIC may reside within a user terminal. In another case, the controller and the storage medium may reside in the user terminal as separate components.

According to an aspect of the present disclosure, an alcohol measurement apparatus may include an inlet formed in a vehicle to absorb a first gas in the vehicle, a shutter provided on the inlet and controlled to be opened or closed to absorb the first gas, an alcohol sensor module provided in a pipe connected with the inlet to measure alcohol from the first gas absorbed via the inlet, and a controller provided in the vehicle to control the shutter to be opened or closed, depending on whether a predetermined measurement condition is met.

The alcohol measurement apparatus may further include a moisture sensor module provided in the pipe connected with the inlet to measure moisture absorbed via the inlet and a drainage part provided in the pipe connected with the inlet and controlled to be opened or closed to drain the moisture which is present on the pipe connected with the inlet. The controller may control the drainage part to be opened or closed, based on the result of measuring moisture.

The controller may determine whether a measurement target in the vehicle is in a drunken state based on the result of measuring the alcohol and may limit starting or driving of the vehicle, if it is determined that the measurement target is in the drunken state.

The controller may remeasure alcohol, if the measurement target corresponds to at least one of a specific behavior or a careless driving state, after a predetermined time, if it is determined that the measurement target is not in the drunken state.

The controller may provide a measurement target in the vehicle with guidance for requesting alcohol measurement, if the vehicle is ignition on or is trying to move in a state in which the vehicle is stopped, and may control the shutter to be opened.

The controller may control the shutter to be opened, if the vehicle is driving, and may measure alcohol from a second gas absorbed via the inlet using the alcohol sensor module.

The controller may determine whether it is possible to measure the alcohol based on an image of a measurement target, the image being captured in the vehicle.

The controller may determine whether it is possible to measure the alcohol based on a face direction and a position of the measurement target in the image.

The controller may determine whether it is possible to measure the alcohol based on a change in face of the measurement target in the image.

The controller may provide the measurement target with an alarm indicating that it is impossible to measure the alcohol, if a predetermined tool or shape is recognized in the image, and may control the shutter to be closed.

According to another aspect of the present disclosure, an alcohol measurement method may include determining whether a predetermined measurement condition is met, controlling a shutter provided on an inlet formed in a vehicle to be opened or closed, depending on whether the predetermined measurement condition is met, and measuring alcohol from a first gas absorbed via the inlet as the shutter is opened or closed, using an alcohol sensor module.

The alcohol measurement method may further include measuring moisture absorbed via the inlet, using a moisture sensor module, and controlling a drainage part for draining the moisture to be opened or closed, based on the result of measuring the moisture.

The alcohol measurement method may further include determining whether a measurement target in the vehicle is in a drunken state based on the result of measuring the alcohol and limiting starting or driving of the vehicle, if it is determined that the measurement target is in the drunken state.

The alcohol measurement method may further include remeasuring alcohol, if the measurement target corresponds to at least one of a specific behavior or a careless driving state, after a predetermined time, if it is determined that the measurement target is not in the drunken state.

The controlling of the shutter to be opened or closed may include providing a measurement target in the vehicle with guidance for requesting alcohol measurement, if the vehicle is ignition on or is trying to move in a state in which the vehicle is stopped, and controlling the shutter to be opened.

The controlling of the shutter to be opened or closed may include controlling the shutter to be opened, if the vehicle is driving, and measuring alcohol from a second gas absorbed via the inlet using the alcohol sensor module.

The determining of whether the predetermined measurement condition is met may include determining whether it is possible to measure the alcohol based on an image of a measurement target, the image being captured in the vehicle.

The determining of whether it is possible to measure the alcohol may include determining whether it is possible to measure the alcohol based on a face direction and a position of the measurement target in the image.

The determining of whether it is possible to measure the alcohol may include determining whether it is possible to measure the alcohol based on a change in face of the measurement target in the image.

The determining of whether it is possible to measure the alcohol may include providing the measurement target with an alarm indicating that it is impossible to measure the alcohol, if a predetermined tool or shape is recognized in the image, and controlling the shutter to be closed.

The present disclosure may minimize an influence by gas (e.g., perfume, carbon monoxide, an alcohol-containing product, or the like), which is not a detection target, thus reinforcing the performance of detecting a drinker in the vehicle.

The present disclosure may prevent sensor pollution and moisture introduction, thus maintaining durability life.

In addition, various effects ascertained directly or indirectly through the present disclosure may be provided.

Hereinabove, although the present disclosure has been described with reference to one or more example embodiments and the accompanying drawings, the present disclosure is not limited thereto, but may be variously modified and altered by those skilled in the art to which the present disclosure pertains without departing from the spirit and scope of the present disclosure claimed in the following claims.

Therefore, the example embodiment(s) of the present disclosure are not intended to limit the technical spirit of the present disclosure, but provided only for the illustrative purpose. The scope of the present disclosure should be construed on the basis of the accompanying claims, and all the technical ideas within the scope equivalent to the claims should be included in the scope of the present disclosure.

What is claimed is:

1. An alcohol measurement apparatus comprising:
an inlet formed in a vehicle and configured to receive a first gas in the vehicle;
a shutter provided on the inlet and configured to open to allow intake of the first gas through the inlet or close to prevent intake of the first gas through the inlet;
an alcohol sensor connected, via a pipe, to the inlet and configured to measure alcohol content of the first gas received via the inlet; and
a control circuit provided in the vehicle and configured to control, based on a predetermined measurement condition, the shutter to open or close.

2. The alcohol measurement apparatus of claim 1, further comprising:
a moisture sensor provided in the pipe and configured to measure moisture content inside the pipe; and
a drain provided in the pipe and configured to:
open to drain moisture in the pipe, or
close to prevent the moisture in the pipe from draining,
wherein the control circuit is further configured to:
control, based on the measured moisture content in the pipe, the drain to open or close.

3. The alcohol measurement apparatus of claim 1, wherein the control circuit is further configured to:
determine whether the measured alcohol content is above a threshold level; and
limit, based on the measured alcohol content being above the threshold level, starting or driving of the vehicle.

4. The alcohol measurement apparatus of claim 1, wherein the control circuit is further configured to:
based on determining that the alcohol content measured at a first time is below a threshold level and detecting, after the first time, a driving pattern of the vehicle that is predetermined to be a careless driving pattern, control the alcohol sensor to measure, at a second time after the first time, second alcohol content of a second gas received via the inlet; and
limit, based on the second alcohol content being above the threshold level, starting or driving of the vehicle.

5. The alcohol measurement apparatus of claim 1, wherein the predetermined measurement condition comprises:
outputting, via a user interface of the vehicle while an ignition of the vehicle is on or the vehicle is operated to move from a stopped position, an alcohol measurement request message; and
controlling the shutter to open.

6. The alcohol measurement apparatus of claim 1, wherein the predetermined measurement condition comprises:
controlling, while the vehicle is moving, the shutter to open; and
controlling the alcohol sensor to measure alcohol content of a second gas received via the inlet.

7. The alcohol measurement apparatus of claim 1, wherein the predetermined measurement condition comprises:
determining, based on an image of a driver in the vehicle, whether it is possible to measure the alcohol content.

8. The alcohol measurement apparatus of claim 7, wherein the control circuit is further configured to:
determine, based on a direction of a face of the driver in the image and a position of the face in the image, whether it is possible to measure the alcohol content.

9. The alcohol measurement apparatus of claim 7, wherein the control circuit is further configured to:
determine, based on a change of a face of the driver in the image, whether it is possible to measure the alcohol content.

10. The alcohol measurement apparatus of claim 7, wherein the control circuit is further configured to:
output, based on detecting a predetermined tool or shape in the image, an alarm indicating that it is impossible to measure the alcohol content; and
control the shutter to close.

11. A method performed by an alcohol measurement apparatus of a vehicle, the method comprising:
determining, based on sensing data of at least one sensor of the vehicle, whether a predetermined measurement condition is met;
controlling, based on whether the predetermined measurement condition is met, a shutter to open or close, wherein the shutter is provided on an inlet connected to an alcohol sensor, and wherein the inlet is formed in the vehicle;
measuring, via the alcohol sensor and based on the shutter being closed, alcohol content of a first gas received via the inlet; and
outputting, based on the measured alcohol content, a signal indicating the measured alcohol content.

12. The method of claim 11, further comprising:
measuring, via a moisture sensor, moisture content in a pipe connecting the inlet to the alcohol sensor; and
controlling, based on the measured moisture content, a drain to open or close.

13. The method of claim 11, further comprising:
determining whether the measured alcohol content is above a threshold level; and
limiting, based on the measured alcohol content being above the threshold level, starting or driving of the vehicle.

14. The method of claim 11, further comprising:
based on determining that the alcohol content measured at a first time is below a threshold level and detecting, after the first time, a driving pattern of the vehicle that is predetermined to be a careless driving pattern, controlling the alcohol sensor to measure, at a second time after the first time, second alcohol content of a second gas received via the inlet; and
limiting, based on the second alcohol content being above the threshold level, starting or driving of the vehicle.

15. The method of claim 11, wherein the predetermined measurement condition comprises:
outputting, via a user interface of the vehicle while an ignition of the vehicle is on or the vehicle is operated to move from a stopped position, an alcohol measurement request message; and
controlling the shutter to open.

16. The method of claim 11, wherein the predetermined measurement condition comprises:
controlling, while the vehicle is moving, the shutter to open; and
measuring, via the alcohol sensor, alcohol content of a second gas received via the inlet.

17. The method of claim 11, wherein the predetermined measurement condition comprises:
determining, based on an image of a driver in the vehicle, whether it is possible to measure the alcohol content.

18. The method of claim 17, wherein the determining of whether it is possible to measure the alcohol content comprises:
determining, based on a direction of a face of the driver in the image and a position of the face in the image, whether it is possible to measure the alcohol content.

19. The method of claim 17, wherein the determining of whether it is possible to measure the alcohol content comprises:
determining, based on a change of a face of the driver in the image, whether it is possible to measure the alcohol content.

20. The method of claim 17, further comprising:
outputting, based on detecting a predetermined tool or shape in the image, an alarm indicating that it is impossible to measure the alcohol content; and
controlling the shutter to close.
